(19) United States
(12) Patent Application Publication
(10) Pub. No.: US 2014/0141066 A1
(43) Pub. Date: May 22, 2014

(54) Encapsulated Ascorbic Acid Composition

(71) Applicants: Edward Pimentel, Port Saint Lucie, FL (US); David Morris Lonardo, Fort Pierce, FL (US)

(72) Inventors: Edward Pimentel, Port Saint Lucie, FL (US); David Morris Lonardo, Fort Pierce, FL (US)

(73) Assignee: LIPO NATURALS LLC, Fort Pierce, FL (US)

(21) Appl. No.: 14/084,102

(22) Filed: Nov. 19, 2013

Related U.S. Application Data
(60) Provisional application No. 61/728,558, filed on Nov. 20, 2012.

(51) Int. Cl. A61K 9/127; A61K 31/341

(52) U.S. Cl. CPC A61K 9/127; A61K 31/341
USPC 424/450; 514/474

(57) ABSTRACT

An encapsulated ascorbic acid composition is a formulation that protects the encapsulated ascorbic acid from unwanted interactions and significantly reduces the likelihood of an unwanted allergic reaction for many users. The encapsulated ascorbic acid composition accomplishes this through the use of sunflower lecithin. Sunflower lecithin is utilized as carrier material that forms the liposomal barrier surrounding a quantity of the ascorbic acid. Sunflower lecithin does not contain common allergens, allowing for increased accessibility of the encapsulated ascorbic acid composition. Additionally, the sunflower lecithin contains a higher quantity of the phospholipid, phosphatidylcholine, compared to other lecithin sources. Phosphatidylcholine is readily absorbed increasing the bioavailability of ascorbic acid in a user. Furthermore, high quantities of phosphatidylcholine form smaller, more stable, and more uniformly shaped liposome resulting in better absorption.

The current application claims a priority to the U.S. Provisional Patent application Ser. No. 61/728,558 filed on Nov. 20, 2012.

### FIELD OF THE INVENTION

The present invention relates generally to an ascorbic acid composition, more specifically to an encapsulated ascorbic acid composition that entraps ascorbic acid within a liposome permitting better absorption following ingestion by a user.

### BACKGROUND OF THE INVENTION

Ascorbic acid, commonly referred to as vitamin C, is a strong anti-oxidant and a cofactor in at least eight essential enzymatic reactions. Vitamin C is required for the synthesis of collagen and norepinephrine, as well as the metabolism of cholesterol. Vitamin C is known to treat or prevent conditions such as scurvy, cardiovascular disease, cancer, age-related macular degeneration, gout, heavy metal toxicity, and diabetes. Although most animal species are able to biosynthesize ascorbic acid from glucose and other sources, humans lack this natural ability and as a result have to acquire it through their diet. Unlike primates (humans), in other species, 160 lb mammal will biosynthesize about 13,000 mg of vitamin C during the day, and many times this under stress conditions such as injury. In order to avoid the various adverse effects of vitamin C deficiency, humans have to consume approximately 90 milligrams of vitamin C each day. Unfortunately, most foods rich in vitamin C are seasonal causing a problem for many individuals. As a result of this situation, many food manufactures fortify their food products with vitamin C. Regardless of fortification, limitations of gut absorption in humans preclude reaching blood levels of vitamin C produced in other species with oral intake alone of the unaltered form of the vitamin.

It is well known that a plurality of processed food products are fortified with vitamin C in order to supplement the recommended daily allowance. Generally, these food products fortified with vitamin C utilize pure ascorbic acid or an ascorbic acid salt. Unfortunately, while the quantity of ascorbic acid consumed in these fortified food products may suffice or exceed the 90 mg daily allowance, the actual amount of ascorbic acid absorbed or utilized by a user may be far lower than anticipated. This situation is due to ascorbic acids antioxidant properties. Ascorbic acid is highly oxidative and can react to various compounds found within food systems prior to ingestion. Ascorbic acid is also sensitive to various environmental factors such as temperature, oxygen concentration, metallic ions, and UV exposure. Resultantly, foods fortified with Vitamin C contain only a fraction of the original fortified quantity due to unwanted interactions prior to ingestion. Another issue experienced with food fortified with pure ascorbic acid occurs following ingestion. Due to its highly reactive nature, a noticeable amount of the ingested pure ascorbic acid is lost to unwanted interactions with digestive enzymes.

In order to reduce degradation and improve the absorption of ascorbic acid a liposomal encapsulation process was developed for use in various food systems. The Liposomal encapsulation process creates a liposomal barrier that surrounds a quantity of ascorbic acid in order to prevent unwanted interaction that results in the degradation of the compound. In addition to preventing degradation of ascorbic acid, the liposomal barrier is comprised of phospholipids which are readily absorbed during digestion by both gut cells and directly into the vascular system. Increased absorption of these phospholipids increases the total absorption of the compound and results in a noticeable increase in its bioavailability. The phospholipids utilized to create the liposomal barrier are derived from various sources but the most common source is soy lecithin. Although soy based products are found in many food products, a significant percentage of the population has a sensitivity or allergy to soy proteins.

One of the most common food allergies are to ingredients and food products derived from soy. The cause of this allergy is due to the excessive activation of an individual's immune system to the presence of soy proteins. This immunoresponse to soy can range from a mild irritation and discomfort, such as red eyes, itchiness, and runny nose, eczema, hives, or an asthma attack, to anaphylaxis, a potentially rapid-onset life threatening condition. As a result of this sensitivity, encapsulated ascorbic acid with a liposomal barrier sourced from soy lecithin would be avoided by a significant portion of the population.

It is therefore the object of the present invention to provide an encapsulated ascorbic acid composition that protects the encapsulated ascorbic acid from unwanted interactions and significantly reduces the likelihood of an unwanted allergic reaction for many users. The encapsulated ascorbic acid composition accomplishes this through the use of sunflower lecithin. Sunflower lecithin is utilized as carrier material that forms the liposomal barrier surrounding a quantity of the ascorbic acid. Sunflower lecithin does not contain common allergens, allowing for increased accessibility of the encapsulated ascorbic acid composition. Additionally the sunflower lecithin contains a higher quantity of the phospholipid, phosphatidylcholine, compared to other lecithin sources. Phosphatidylcholine is readily absorbed increasing the bioavailability of ascorbic acid in a user. Furthermore, high quantities of phosphatidylcholine form smaller more stable, and more uniformly shaped liposome resulting in better absorption.

### BRIEF DESCRIPTIONS OF THE DRAWINGS

FIG. 1 is a partial cross sectional view of the encapsulated ascorbic acid composition displaying a cross section of the carrier material surrounding an intact ascorbic acid solution.

FIG. 2 is an enhanced perspective view displaying the lipid bilayer structure formed by the phospholipids of lecithin.

FIG. 3 is a chemical line drawing representing the structure and composition of phosphatidylcholine.

### DETAIL DESCRIPTIONS OF THE INVENTION

All illustrations of the drawings are for the purpose of describing selected versions of the present invention and are not intended to limit the scope of the present invention.

The present invention is an encapsulated ascorbic acid composition that is specially designed to increase the bioavailability of ascorbic acid in a subject. The present invention accomplishes this by coating ascorbic acid in a carrier material 1 that functions as a barrier between the ascorbic acid and its surroundings. The carrier material 1 protects the ascorbic acid from unwanted interactions with digestive enzymes and permits absorption into fatty tissue increasing cellular concentration of ascorbic acid. In the current embodiment of the present invention the encapsulated ascorbic acid comprises the carrier material 1 and an ascorbic acid solution 3. The ascorbic acid solution 3 is an aqueous solution containing ascorbic acid. The ascorbic acid solution 3 facilitates the encapsulation of the ascorbic acid in the carrier material 1 through a liposome encapsulation process.

The ascorbic acid solution 3 comprises distilled water and ascorbic acid. The ascorbic acid is the active agent that is dissolved in the distilled water in order to facilitate encapsulation by the carrier material 1. The distilled water is the preferred solvent for dissolving the ascorbic acid due to its reduced mineral ion concentration that could potentially react with the ascorbic acid. The ascorbic acid solution 3 is a saturated solution under conditions of standard temperature and pressure (STP). Although the ascorbic acid solution 3 can be a super saturated solution, the conditions necessary to achieve this would require an increase in temperature resulting in the degradation of the dissolved ascorbic acid. In the current embodiment of the present invention, ascorbic acid is found ranging between 37 wt % to 47 wt % of the encapsulated ascorbic acid composition. The provided range for ascorbic acid is in accordance with the relative weight ratio to lecithin 2, wherein lecithin 2 is present in the encapsulated ascorbic acid composition at a 5.6:1 ratio by weight relative to ascorbic acid.

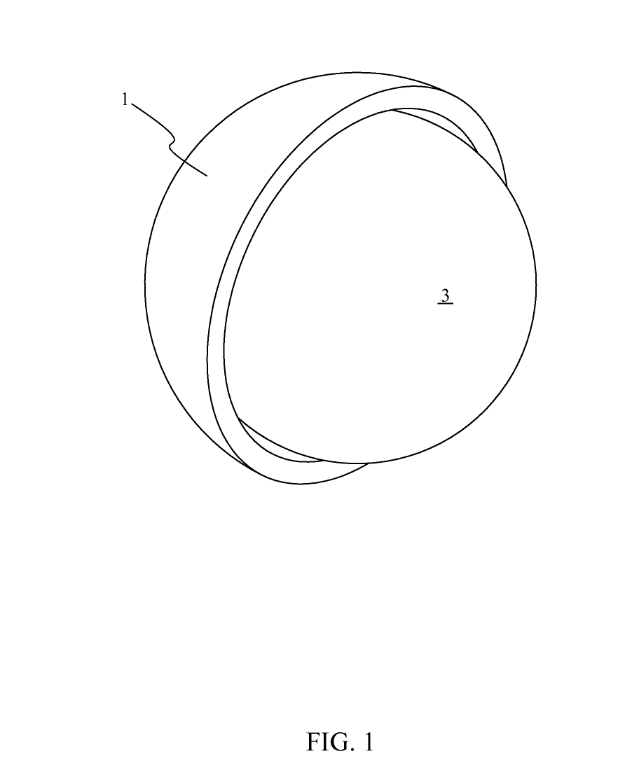

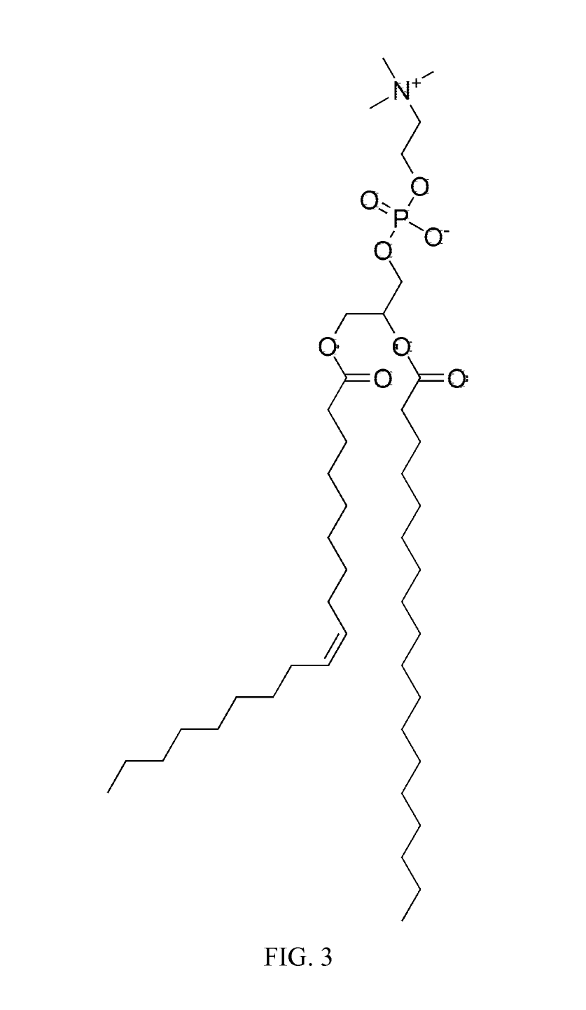

Referencing FIG. 1, the carrier material 1 functions as the encapsulating material that functions as a barrier protecting the ascorbic acid from reacting prematurely. The carrier material 1 provides a delivery mechanism for the ascorbic acid that permits absorption into fatty tissue. In the current embodiment of the present invention, the carrier material 1 comprises lecithin 2. Lecithin 2 is a fatty substance derived from plant or animal tissue that is comprised of a plurality of different phospholipids and lipids. The lecithin 2 utilized as the carrier material 1 is derived from sunflowers. The sunflower derived lecithin 2 is utilized primarily for its lack of common allergens. Most lecithin 2 is generally derived from a group of foods that is associated with some of the most common food allergies. This group comprises eggs, milk, and soy. Through the use of sunflower derived lecithin 2, the present invention is able to avoid introducing dairy and soy proteins that cause food based allergic reactions into the encapsulated ascorbic acid composition. Referencing FIG. 3, the sunflower derived lecithin 2 is known to contain a higher percentage of the phospholipid, phosphatidylcholine compared to egg or milk derived lecithin 2. The high percentage phosphatidylcholine provides improved liposomal formation facilitating the encapsulation of the ascorbic acid solution 3. In the current embodiment of the present invention, lecithin 2 is found ranging between 53 wt % to 63 wt % of the encapsulated ascorbic acid composition. The provided range for lecithin 2 is in accordance with the relative weight ratio to ascorbic acid, wherein lecithin 2 is present in the encapsulated ascorbic acid composition at a 5.6:1 ratio by weight relative to ascorbic acid.

TABLE 1
Encapsulated Ascorbic Acid Composition

| | wt % of | |
|---|---|---|
| | composition | Active Agent |
| Ascorbic acid solution | 37 to 47 wt % | Ascorbic Acid |
| Carrier material | 53 to 63 wt % | Lecithin |

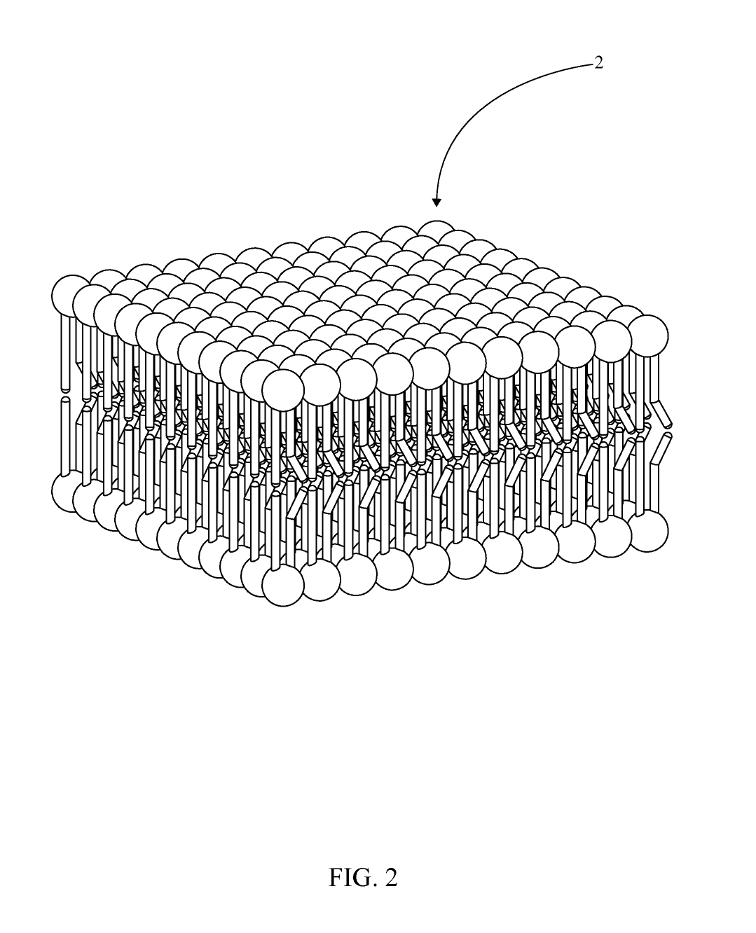

The ascorbic acid solution 3 is encapsulated by lecithin 2 forming liposomes. Liposomes are fluid filled vesicles that comprise a membranous exterior surrounding an aqueous volume as a core. In the current embodiment of the present invention, the aqueous volume is the ascorbic acid solution 3 while the membranous exterior is formed by lecithin 2. Lecithin 2 is able to form a membranous exterior due to containing a high quantity of phospholipid. Referencing FIG. 2, phospholipids are amphiphilic allowing them to align in aqueous environments forming a lipid bilayer where the exterior surface comprises the hydrophilic polar heads and the interior comprises the hydrophilic hydrocarbon tails. The liposomes formed from lecithin 2 consist mostly of the phospholipid, phosphatidylcholine. Phosphatidylcholine is essential in the formation of cellular membranes and is readily absorbed during digestion, facilitating the transport of the encapsulated ascorbic acid solution 3. Additionally, by comprising mostly of phosphatidylcholine, liposomes derived from lecithin 2 are more stable and can be made relatively uniform in size. In the current embodiment of the present invention, the liposomes are formed with a particle size diameter ranging between 100-400 nm. The aforementioned particle size diameter range enables the liposomes to be more easily absorbed during digestion. In order to size the liposomes with the aforementioned particle size diameter range, the liposomes are formed through a liposome encapsulation process.

The ascorbic acid solution 3 is encapsulated in the carrier material 1 through a liposome encapsulation process. The liposome encapsulation process generally comprises the steps of preparing the carrier material 1, preparing the ascorbic acid solution 3, combining the lecithin 2 with the ascorbic acid solution 3, and sizing the liposomes. The step of preparing the carrier material 1 is a purification step that separates unwanted lipid or phospholipids from the lecithin 2. The preparation of the ascorbic acid solution 3 is a step that dissolves an amount of ascorbic acid in solution. The step of combining the lecithin 2 and the ascorbic acid solution 3 forms a lipid suspension. The step of sizing the liposomes disrupts the suspension and forms liposomes within a desired particle size diameter range.

The step of preparing the carrier material 1 is a purification step that separates unwanted lipids and phospholipids from the carrier material 1. This separation provides a more homogeneous phospholipid composition of the carrier material 1 allowing for the formation of small, stable, and uniformly sized liposomes. The separation of unwanted lipids and phospholipids can be accomplished through any separation technique or protocol that does not negatively impact or chemically alter the intended carrier material 1. It should be noted that in the current embodiment of the present invention, the high quantity of phosphatidylcholine found in lecithin 2 does not necessitate the preparation step, as the step would not significantly improve the formation of small, stable, and uniformly sized liposomes. In the current embodiment the preparation step would be for standardization and optimization in order to ensure the consistency but could be omitted completely if desired.

The step of preparing the ascorbic acid solution 3 forms a saturated ascorbic acid solution 3 from ascorbic acid and distilled water. The preparation step dissolves ascorbic acid in distilled water until the solution is saturated under standard conditions. The solubility of pure ascorbic acid in distilled water at STP is 330 g/L. The prepared ascorbic acid solution 3 is utilized as a stock solution that is aliquoted in combination with a quantity of the carrier material 1.

The step of combining the lecithin 2 and the ascorbic acid solution 3 forms a lipid suspension. In the combination step, an amount of the ascorbic acid solution 3 is added to an amount of the lecithin 2 to form a mixture. The amount of the ascorbic acid solution 3 added to the amount of lecithin 2 is provided according to the 5.6:1 ratio by weight, where lecithin 2 is present in three times the quantity to pure ascorbic acid. Following the addition of the ascorbic acid solution 3, the mixture is agitated in order to ensure proper hydration of the lecithin 2. Agitation of the mixture can occur for several hours and is stopped when it is determined the lecithin 2 has been properly hydrated. The lipid suspension formed by agitation of the mixture is an emulsion containing variably sized liposomes.

The step of sizing the liposomes disrupts the suspension and forms liposomes within a desired particle size diameter range. In order to properly size liposomes from the lipid suspension, a mechanical dispersion method is utilized. The mechanical dispersion method is a mechanical means of disrupting the lipid suspension in order to form particularly sized liposomes. In the current embodiment of the present invention, the mechanical dispersion method is provided as either an extrusion process or a sonification process. The extrusion process is a mechanical process that uses kinetic energy to form liposomes. The sonification process utilizes sonic energy to further agitate the lipid suspension fracturing variably sized liposomes so that they are more uniformly sized.

The extrusion process extrudes the lecithin 2 and the ascorbic acid solution 3 through an ejection tube at high velocities towards a target plate. Upon impacting the target plate, the ascorbic acid solution 3 combines with the lecithin 2 forming liposomes with an average particle size diameter ranging between 100 nm-400 nm. In an embodiment of the present invention, the extrusion process is elected as the mechanical dispersion method. The extrusion process is selected due to being a simple, rapid, and reproducible process that is well known for forming liposomes.

The sonification process utilizes an ultrasonic mixer to sonicate liposomes from the lipid suspension. The ultrasonic mixer vibrates the lipid suspension contained within a vessel at a particular frequency fracturing the lipid bilayer of the variably sized liposomes. The fractured portions of the lipid bilayer are uniform in size and recombine into smaller uniformly sized liposomes with an average particle size diameter ranging between 100 nm-400 nm. In an embodiment of the present invention, the sonification process can be selected due to forming smaller and more uniformly sized liposomes in comparison to the extrusion process.

Although the present invention specifically mentions the sonification process and the extrusion process, additional mechanical dispersion processes may be utilized. It should be noted that the present invention can utilize any mechanical dispersion process that does not degrade the ascorbic acid but is also able to form liposomes with an average particle size diameter ranging between 100 nm-400 nm.

In an embodiment of the present invention, the ascorbic acid solution 3 comprises a preservative. The preservative is an auxiliary agent that improves the shelf life of the encapsulated ascorbic acid composition. The preservative would inhibit or significantly limit microbial activity and mold growth. It should be noted that the preservative could be provided as any soluble agent that would not significantly affect the integrity and size of the liposome, nor the solubility of the ascorbic acid but provide the necessary properties to extend the shelf life of the encapsulated ascorbic acid composition. In an embodiment, the preservative is alcohol and is provided as being no more than 10% by volume of the ascorbic acid solution 3.

Alcohol is elected as the preservative for its ability to reduce the water activity a_{w} of an aqueous solution as well as for its ability to disrupt cellular metabolism. Water activity a_{w} is an indicator of chemical or microbial kinetics and is calculated from the partial pressure of water in a solution compared to the vapor pressure of pure water. A high a_{w} value signifies increased susceptibility to microbial growth while a lower value signifies decreased susceptibility to microbial growth. Alcohols ability to lower water activity within a solution is supported by Raoult's law. Additionally, alcohol has antimicrobial properties partly due to its amphiphilic nature allowing it to traverse the plasma membrane of a microbial organism. Once inside, the plasma membrane alcohol inhibits DNA, RNA, and protein biosynthesis disrupting essential metabolic functions.

The encapsulated ascorbic acid composition functions as a nutritional supplement that facilitates the absorption of ascorbic acid. A user would orally administer the encapsulated ascorbic acid composition. The encapsulated ascorbic acid composition can be administered alone or in conjunction within another consumable good. Following ingestion, a percentage of the encapsulated ascorbic acid composition would be absorbed by the users gut, initially increasing the amount of ascorbic acid found in the user's blood. Another percentage of the encapsulated ascorbic acid composition would be absorbed and stored within the fatty cellular tissues of the subject. The absorbed encapsulated ascorbic acid composition would slowly be released as the carrier material 1 surrounding the ascorbic acid solution 3 is metabolized. With the carrier material 1 metabolized, the ascorbic acid solution 3 is released and absorbed into the blood stream. The absorbed ascorbic acid increases the percentage of ascorbic acid in the blood stream compounding the initially absorbed ascorbic acid.

In the current embodiment of the present invention, the ascorbic acid solution 3 comprises distilled water and ascorbic acid. Ascorbic acid is commonly referred to as vitamin C. Ascorbic acid is a strong anti-oxidant and a cofactor in at least eight essential enzymatic reactions. Vitamin C is required for the synthesis of collagen and norepinephrine, as well as the metabolism of cholesterol. Vitamin C has been shown in published studies to treat or prevent conditions such as cancer (“High-Dose Vitamin C (PDQ®)—National Cancer Institute.”, The National Cancer Institute. www.cancer.govicancertopics/pdq/cam/highdosevitaminc/healthprofessional 7 Aug. 2013), scurvy, cardio vascular disease, age-related macular degeneration, gout, heavy metal toxicity, and diabetes (“Vitamin C: Medline Plus Medical Encyclopedia.” National Institute of Health www.nlm.nih.gov/medlineplus/ency/article/002404.htm 27 Aug. 2012). Although the ascorbic acid is described as being vitamin C, it should be noted that conjugates, isomers, and enantiomers of ascorbic acid can also be described as Vitamin C. Resultantly, ascorbic acid can be specified as or substituted for L-ascorbic acid and L-ascorbate, as well as a mineral salt of ascorbic acid, or an ester of ascorbic acid.

In the current embodiment of the present invention, the preparation of the ascorbic acid solution 3 may be improved through the use of future technologies that may allow for pressurization of a cooled medium in order to super saturate ascorbic acid solution 3. By super saturating the ascorbic acid solution 3 the encapsulated ascorbic acid composition would contain a higher concentration of ascorbic acid within each liposome.

In the current embodiment of the present invention, the carrier material 1 comprises lecithin 2 derived from sunflowers. Lecithin 2 derived from sunflowers is a fatty acid substance that is hydrolyzed or fractionated from sunflower seed oil. Although the current embodiment of the present invention does not specifically mention if the sunflower lecithin 2 is hydrolyzed or fractionated from sunflower seed oil, it should be noted that the encapsulated ascorbic acid composition may comprise hydrolyzed sunflower lecithin 2 or fractionated sunflower lecithin 2, as well as any combination thereof.

Although the invention has been explained in relation to its preferred embodiment, it is to be understood that many other possible modifications and variations can be made without departing from the spirit and scope of the invention as hereinafter claimed.

What is claimed is:

1. An encapsulated ascorbic acid composition comprises:
a carrier material;
an ascorbic acid solution;
the carrier material comprises lecithin;
the ascorbic acid solution comprises distilled water and ascorbic acid;
lecithin being derived from sunflowers; and
the ascorbic acid solution being encapsulated by lecithin forming a liposome.

2. The encapsulated ascorbic acid composition in claim 1 wherein the lecithin is found ranging between 53 wt % to 63 wt % of the composition.

3. The encapsulated ascorbic acid composition in claim 1 wherein the ascorbic acid is found ranging between 37 wt % to 47 wt % of the composition.

4. The encapsulated ascorbic acid composition in claim 1 wherein, the lecithin is present in a 5.6:1 ratio by weight relative to the ascorbic acid.

5. The encapsulated ascorbic acid composition in claim 1 wherein, the ascorbic acid solution comprises an amount of a preservative.

6. The encapsulated ascorbic acid composition in claim 5 wherein, the preservative comprises Ethanol (EtOH).

7. The encapsulated ascorbic acid composition in claim 1 wherein, the liposomes being formed by a sonification process.

8. The encapsulated ascorbic acid composition in claim 1 wherein, the liposomes being formed by an extrusion process.

9. The encapsulated ascorbic acid composition in claim 1 wherein, the liposome having a particle size diameter ranging 100-400 nm.

10. An encapsulated ascorbic acid composition comprises:
a carrier material;
an ascorbic acid solution;
the carrier material comprises lecithin;
the ascorbic acid solution comprises distilled water and ascorbic acid;
lecithin being derived from sunflowers;
the ascorbic acid solution being encapsulated by lecithin forming a liposome;
lecithin is found ranging between 53 wt % to 63 wt % of the composition;
the ascorbic acid is found ranging between 37 wt % to 47 wt % of the composition;
the lecithin is present in a 5.6:1 ratio by weight relative to the ascorbic acid; and
the liposome having a particle size diameter ranging 100-400 nm.

11. The encapsulated ascorbic acid composition in claim 9 wherein, the ascorbic acid solution comprises an amount of a preservative.

12. The encapsulated ascorbic acid composition in claim 11 wherein, the preservative comprises Ethanol (EtOH).

13. The encapsulated ascorbic acid composition in claim 9 wherein, the liposomes being formed by a sonification process.

14. The encapsulated ascorbic acid composition in claim 9 wherein, the liposomes being formed by an extrusion process.

15. An encapsulated ascorbic acid composition comprises:
a carrier material;
an ascorbic acid solution;
the carrier material comprises lecithin;
the ascorbic acid solution comprises distilled water and ascorbic acid;
lecithin being derived from sunflowers;
the ascorbic acid solution being encapsulated by lecithin forming a liposome;
lecithin is found ranging between 53 wt % to 63 wt % of the composition;
the ascorbic acid is found ranging between 37 wt % to 47 wt % of the composition;
the lecithin is present in a 5.6:1 ratio by weight relative to the ascorbic acid; and
the liposome having a particle size diameter ranging 100-400 nm.

16. The encapsulated ascorbic acid composition in claim 15 wherein, the ascorbic acid solution comprises an amount of a preservative, where the preservative comprises Ethanol (EtOH).

17. The encapsulated ascorbic acid composition in claim 15 wherein, the liposomes being formed by a sonification process.

18. The encapsulated ascorbic acid composition in claim 15 wherein, the liposomes being formed by an extrusion process.
